(19) United States
(12) Patent Application Publication
(10) Pub. No.: US 2021/0263120 A1
(43) Pub. Date: Aug. 26, 2021

(54) METHOD OF PERSONALIZED TREATMENT FOR CARDIOMYOPATHY AND HEART FAILURE AND ASSOCIATED DISEASES BY MEASURING EDEMA AND CACHEXIA/SARCOPENIA

(71) Applicant: ARIZONA BOARD OF REGENTS ON BEHALF OF THE UNIVERSITY OF ARIZONA, Tucson, AZ (US)

(72) Inventors: Guy Reed, Phoenix, AZ (US); Inna Gladysheva, Phoenix, AZ (US); Ryan Sullivan, Phoenix, AZ (US); Radhika Mehta, Phoenix, AZ (US)

(21) Appl. No.: 17/313,904

(22) Filed: May 6, 2021

Related U.S. Application Data
(63) Continuation-in-part of application No. PCT/US19/60047, filed on Nov. 6, 2019.
(60) Provisional application No. 62/756,409, filed on Nov. 6, 2018.

(51) Int. Cl. G01R 33/48; A61B 5/00; A61B 5/055

(52) U.S. Cl. CPC G01R 33/4828; A61B 5/7275; A61B 5/055; A61B 5/0036; A61B 5/4839

(57) ABSTRACT

Methods of utilizing quantitative magnetic resonance (QMR) to non-invasively determine and quantify body fluid dynamics [e.g., extracellular water and total water levels] and body compositions (e.g., lean muscle mass and fat mass levels). These levels or changes therein are used as biomarkers to diagnose, prognose, tailor and monitor treatment for conditions that cause or caused by ECW retention (edema), lean muscle mass loss (sarcopenia), and/or fat loss (cachexia), including heart failure (HF) and HF-associated and HF-non-associated edema/sarcopenia/cachexia, kidney disease, liver disease, and other HF complications. Body fluid dynamics and body compositions measurements obtained from QMR technology are used as biomarkers to help a physician's assessment to determine the prognosis and personalized treatment strategies that are tailored for individual patients with heart dysfunction and/or HF, HF-associated conditions, and/or other conditions that cause or caused by edema, sarcopenia, and/or cachexia.

### CROSS-REFERENCES TO RELATED APPLICATIONS

This application is a continuation-in-part and claims benefit of PCT Application No. PCT/US19/60047, filed Nov. 6, 2019, which claims priority to U.S. Provisional Patent Application No. 62/756,409, filed Nov. 6, 2018, the specifications of which are incorporated herein in their entirety by reference.

### BACKGROUND OF THE INVENTION

A number of conditions cause extracellular water (ECW) retention (e.g., edema), lean muscle mass loss (e.g., sarcopenia), and fat loss (e.g., cachexia). Such conditions include heart failure (HF), HF-associated and HF-non-associated edema/sarcopenia/cachexia, kidney disease, liver disease and muscular dystrophy. As such HF has broad systemic effects on organs such as the lungs, kidneys, and liver, which are not emphasized by traditional HF classification systems.

### Field of the Invention

The present invention relates to a method of utilizing quantitative magnetic resonance (QMR) (for example, EchoMRI™ or nuclear magnetic resonance-magnetic resonance imaging, NMR-MRI) technology to assess body fluid dynamics such as ECW (e.g., excess ECW/edema) and total water and body composition biomarkers such as lean muscle mass, and fat mass to diagnose, prognose, tailor and monitor treatment conditions that cause or caused by ECW retention (edema), and/or lean muscle mass loss (sarcopenia), and/or fat loss (cachexia). In particular, this invention uses measurements (biomarkers) obtained from QMR technology to help determine the prognosis and personalized treatment strategy for patients with heart dysfunction and/or HF, HF-associated conditions, or other conditions that cause or caused by ECW retention, and/or lean muscle mass loss, and/or fat loss. With a physician's assessment, precision therapies then can be tailored to individuals for personalized treatment of heart dysfunction, HF or related diseases.

### Background Art

Increases in ECW (or the water outside the cell; ECW retention) causes excess weight and swelling in the limbs, pleural effusion, lung and peripheral edema defining transition from heart dysfunction to clinical HF. It can be an early sign of an imbalance in the body including imbalances in hormone, protein, sodium, potassium, magnesium, and pH. Several conditions cause or are caused by ECW retention including HF. Loss of lean muscle/sarcopenia and fat/cachexia are reliable indicators of poor HF outcomes. Current treatment guidelines from HF organizations do not address diagnosis, management, or therapeutics for either condition.

Heart failure (HF) has many causes and HF progression is affected by various pathways, including the sympathetic nervous (SN) system, the renin angiotensin-aldosterone-system (RAAS) and the natriuretic peptide (NP) system. Activation of the SN and RAA systems is associated with extracellular fluid and sodium retention (edema), left ventricular dysfunction, and cardiac dilation. The NP system promotes diuresis, natriuresis and vasodilation, which acts to counter the SN-RAA systems.

Patients with HF are often treated broadly with the same medications, based on large randomized clinical trials. These trials homogenize individual differences, although it is widely known that HF has many etiologies and patients show differences in their biomarker profiles. HF has many causes and progression is affected by various pathways. Current treatment solutions recommend diuretics, dobutamine, and therapeutics against angiotensin, aldosterone or neprilysin, without proper assessment of biomarkers such as changes in ECW retention (e.g., edema), lean muscle mass loss (e.g., sarcopenia), and fat loss (e.g., cachexia).

Individual HF patients often have variable left ventricular function and may progress at different clinical rates. It is also increasingly recognized that there are disparities in treatment outcomes related to a variety of factors such as sex, race, geographic location, disease etiology, and genetics causes. Consequently, there remains a need for improving HF diagnosis, prognosis and treatment. This necessitates a solution to identify specific biomarkers and aid in targeted treatment plans.

Improving the treatment of HF necessitates a precision medicine approach, which requires a two-prong technique that relies on 1) identification of specific biomarkers, which allow for earlier diagnoses and classification of a disease profile and, 2) a targeted treatment plan for the individual based on their specific profile (e.g., personalized treatment strategy).

Despite HF being widely studied, technology is rather limited regarding HF diagnostics and individualized treatment options for prevention and clinical symptoms. Typically, treatments are physician-dependent and HF-associated edema, sarcopenia (muscle mass loss)/cachexia (wasting syndrome) are difficult to target. Only a few companies are developing QMR machines for whole body assessment that noninvasively measure whole body fat, lean muscle mass, free water (ECW), and total water masses in live conscious animals, including humans.

Other technologies to measure edema may include MRI, bioimpedance analysis (BIA), bioimpedance spectroscopy (BIS), or a baseline edema measuring device which is a volumetric measuring device which utilizes water displacement. These systems require specialized training, prolonged measuring times, and are not widely available for longitudinal patient monitoring during disease identification, progression or monitoring. QMR compared to the above mentioned modalities is more sensitive and thus able to detect changes in ECW and/or lean muscle mass, and/or fat mass prior to reaching critical edema stage (the final outcome of ECW retention) and/or cachexia (final outcome of body fat loss>5-6%) and/or sarcopenia (final outcome of body lean muscle mass loss) and therefore can be used to precisely scan, monitor, and treat individuals at risk or with altered ECW (e.g., edema), fat mass (e.g., /cachexia), and/or lean muscle mass (e.g., sarcopenia).

### BRIEF SUMMARY OF THE INVENTION

It is an objective of the present invention to provide methods that allow for personalized treatment for cardiomyopathy and HF and associated diseases by non-invasively measuring edema and cachexia/sarcopenia, as specified in the independent claims. Embodiments of the invention are given in the dependent claims. Embodiments of the present invention can be freely combined with each other if they are not mutually exclusive.

The present invention features a new method of use of QMR, measuring ECW, total water, muscle lean mass, and fat mass, for diagnostic, prognostic, and treatment tailoring and monitoring purposes for heart-related diseases, HF-associated and HF-non-associated edema/sarcopenia/cachexia, kidney disease, liver disease and muscular dystrophy. Excess ECW (e.g. edema), muscle/lean mass loss (e.g., sarcopenia), and fat loss (e.g., cachexia) can be measured, aiding in the prognosis and progression of patients with heart dysfunction, at risk for HF, and HF. As there remains a need for an individualized/personalized treatment of heart dysfunction/cardiomyopathy and HF based on identifying specific biomarkers, physicians will be able to use the present invention to recommend personalized treatments (i.e., precision medicine), a feature commonly unavailable to patients who at risk for HF or with HF. The present invention provides a solution addressing the industry need as earlier diagnoses and classification of disease profiles allow physicians to conduct individualized targeted treatment.

One of the unique and inventive technical features of the present invention is using QMR technology for personalized medicine. In particular, this invention utilizes the resultant changes in biomarkers including ECW, lean muscle body mass, and body fat mass levels (e.g., as measured by EchoMRI™). The resultant changes are indicators for personalized treatment strategies, including personalized diagnosis, prognosis, treatment monitoring, and disease development and progression monitoring for conditions that cause or caused by ECW retention (e.g., edema), lean muscle mass loss (e.g., sarcopenia), and/or fat loss (e.g., cachexia). Without wishing to limit the invention to any theory or mechanism, it is believed that using QMR to non-invasively evaluate ECW, lean muscle body mass, and body fat mass levels and changes thereof advantageously provides for personalized treatment approaches for conditions that cause or caused by ECW retention/edema, and/or lean muscle mass loss/sarcopenia, and/or fat loss/cachexia, including HF-related conditions.

Further, the prior art teach away from using non-invasive quantification of ECW, lean muscle mass, and fat mass for routine monitoring of HF patients because of the lack of sensitivity of available modalities to measure and distinguish critical changes necessary to detect clinically evident and non-evident symptoms of HF (e.g., edema, sarcopenia, cachexia). For example, MRI alone, BIA, BIS can measure ECW and dual-energy X-ray absorptiometry (DXA or DEXA, commonly referred to as bone mineral density scanning), MRI, and computed tomography can measure muscle mass and fat mass but are not sensitive enough to measure or distinguish critical values of ECW prior to reaching edema or critical values of lean muscle mass and fat mass before reaching sarcopenia and cachexia, respectively. However, the present invention applies a new use of QMR, which measures and quantifies ECW, lean muscle mass, and fat mass and surprisingly was sensitive enough to detect changes in ECW, lean muscle mass, and/or fat mass prior to reaching clinically evident critical stages of edema, cachexia, and/or sarcopenia. QMR provides a quick and non-invasive method to objectively detect and quantify by weight, not by image, as is the case of prior art technologies. For example, QMR can measure ECW throughout the disease progression prior to a patient developing systemic edema. Serial QMR measurements permit the detection of body fat and muscle losses, which may be masked by simply following body weights as currently recommended for management of HF patients. Therefore, the present invention identifies changes in ECW, lean muscle mass, and fat mass associated with cardiac cachexia and sarcopenia in late stages of HF.

Therefore, the present invention can be used to precisely scan, monitor, and treat individuals at risk or with altered ECW (e.g., edema), fat mass (e.g. cachexia), and/or lean muscle mass (e.g., sarcopenia) as compared to prior technologies (e.g., MRI alone, BIA, BIS, DEXA, computed tomography). As such, the advantages of the present invention are operational ease, recording speed, reproducibility, and accurate measurements that do not require interpretation of images. The present invention is a useful modality for monitoring human disease progression and the efficacy of therapy for HF.

The present invention features methods for determining diagnosis and prognosis of a patient (the patient can be symptomatic or asymptomatic for HF-related conditions) who is suspected or has a condition that causes or caused by ECW retention/edema, lean muscle mass loss/sarcopenia, and/or fat loss/cachexia, including HF and HF-related conditions. In preferred embodiments, the method comprises first using QMR to measure at least one or more levels of: 1) ECW/edema; 2) muscle/lean mass/sarcopenia; and/or 3) fat/cachexia of the patient. The patient is then diagnosed and/or prognosed based on the measured ECW content; total water content; muscle/lean mass, and/or fat mass. A personalized treatment approach for the patient is then determined using the diagnosis and/or prognosis based on the measured fluid dynamics (e.g., total water, ECW) and body compositions (e.g., lean muscle mass, fat mass) as well as the specific clinicopathologic characteristics (e.g., sex, age, history of diet, smoking, exercise, and HF-related conditions) of the patient

The present invention further features a method for treating a patient that has a condition that causes or is caused by ECW retention/edema, lean muscle mass loss/sarcopenia, and/or fat loss/cachexia, including HF and HF-related conditions. In preferred embodiments, the method comprises first using QMR to measure at least one or more levels of: 1) ECW/edema; 2) muscle/lean mass/sarcopenia; and/or 3) fat/cachexia of the patient. A personalized treatment approach for the patient is then determined using the prognosis based on the measured fluid dynamics (e.g., total water, ECW) and body compositions (e.g., lean muscle mass, fat mass) as well as the specific clinicopathologic characteristics (e.g., sex, age, history of diet, smoking, exercise, and HF-related conditions) of the patient. The invention features an additional step of administering a therapy based on agents that specifically interfere with causes of edema, sarcopenia, and/or cachexia, including renin/pro-renin plasma activity and/or renin/pro-renin interaction with cardiomyopathy and HF-related conditions. Other interventions (e.g., physically draining a cavity, exercising or nutritional alterations) may also be used to treat the patient.

The present invention also features a method for monitoring a personalized treatment for a condition that causes or is caused by ECW retention/edema, lean muscle mass loss/sarcopenia, and/or fat loss/cachexia, including HF and HF-related conditions. In preferred embodiments, the method comprises first using QMR to measure at least one or more levels of: 1) ECW/edema; 2) muscle/lean mass/sarcopenia; and/or 3) fat/cachexia of the patient. A personalized treatment approach for the patient is then determined using the prognosis based on the measured fluid dynamics (e.g., total water, ECW) and body compositions (e.g., lean muscle mass, fat mass) measured at the different timepoints (e.g., two to seven days post-diagnosis, one month post diagnosis, three months post diagnosis, or >three months post-diagnosis as compared to baseline measurements at time of diagnosis) based on the prognosis determining risk of progression of death. The personalized treatment or approach for the patient can then be changed based on the differences of the body fluid dynamics and compositions as well as the specific clinicopathologic characteristics (e.g., sex, age, diet, smoking status, prior history of HF-related conditions) of the patient. In some embodiments, the invention features an additional step, for example, changing the therapy or requiring additional monitoring or another intervention if the patient is progressing. In other embodiments, if the patient is not progressing or responding to treatment, for example, having a decreased ECW content and increased lean muscle mass, the personalized treatment may not be changed and/or the frequency of monitoring may be decreased.

The present invention further features a method for monitoring disease progression of conditions that causes or is caused by ECW retention/edema, lean muscle mass loss/sarcopenia, and/or fat loss/cachexia, including HF and HF-related conditions. In preferred embodiments, the method comprises first using QMR to measure at least one or more levels of: 1) ECW/edema; 2) muscle/lean mass/sarcopenia; and/or 3) fat/cachexia of the patient. The patient is then prognosed and a risk of progression or death is determined based on the measured fluid dynamics (e.g., total water, ECW) and body compositions (e.g., lean muscle mass, fat mass) at the different timepoints (e.g., two to seven days post-diagnosis, one month post diagnosis, three months post diagnosis, or >three months post-diagnosis as compared to baseline measurements at time of diagnosis). A personalized treatment approach for the patient can then be developed (if patient hasn't started treatment) or changed based on the differences of the measured fluid dynamics (e.g., total water, ECW) and body compositions (e.g., lean muscle mass, fat mass) as well as the specific clinicopathologic characteristics (e.g., sex, age, diet, exercise, smoking history, prior history of HF-related conditions) specific to the patient. In some embodiments, the invention features an additional step, for example, starting a therapy or changing the therapy or requiring additional or more frequent monitoring or another intervention if the patient is progressing or having further increases in plasma renin activity. In other embodiments, if the patient is not progressing and having decreases in plasma renin activity, the personalized treatment may not be changed and/or the frequency of monitoring may be decreased.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A, 1B, 1C, and 1D show heart failure (HF) stages, study design, and effects of aliskiren, a direct renin inhibitor (DRI). FIG. 1A shows a schematic overview of the natural history of HF progression, biomarker changes, and experimental design, in an established model of dilated cardiomyopathy (DCM) in female mice. Female mice with DCM begin to show declines in heart systolic function (ejection fraction; EF) and increases in plasma renin activity around 7 weeks of age (Stage B HF), which is prior to the development of progressive edema (Stage C HF), further declines in systolic function, rises in atrial/B-type natriuretic peptide (ANP/BNP) and death. Mice with DCM were randomly treated with aliskiren (DCM+DRI) or nothing (DCM+vehicle) in drinking water (see Examples Section). Vertical hash-mark lines indicate time points for measurement of body composition, while echocardiography and blood-tissue collection were completed at 90 days. FIG. 1B shows the impact of aliskiren treatment on renin plasma activity at 90 days. FIG. 10 shows the impact of aliskiren on angiotensin II (Ang II) at 90 days. FIG. 1D shows the impact of aliskiren on aldosterone levels at 90 days. The number of DCM mice is indicated. For reference in FIGS. 1B-1D, values for wild-type (WT) littermates are shown as a dashed line (n=4). Data analyzed with one-way ANOVA and represented as mean±SE. Not significant (NS), ⁺⁺p<0.01, ⁺⁺⁺p<0.001 (solid circle, WT vs. DCM+vehicle; solid square, WT vs. DCM+DRI), ***p<0.001 (DCM+vehicle vs. DCM+DRI).

FIGS. 2A, 2B, 2C, 2D, 2E, 2F, and 2G show that direct renin inhibitor (DRI) treatment significantly improves survival and systolic function in mice with dilated cardiomyopathy (DCM). FIG. 2A shows Kaplan-Meier survival curves of control mice with DCM (DCM+vehicle, red, n=13 deaths+8 censored) vs. DCM mice treated with DRI (DCM+DRI, black, n=21 deaths+8 censored). WT (n=4) values are provided for reference. FIG. 2B shows short axis m-mode examples of DCM+vehicle and DCM+DRI treated mice at 90 days of age. FIG. 2C shows left ventricular systolic function measured as ejection fraction (EF) between DCM+vehicle and DCM+DRI mice. [EF, wildtype (WT)=62.8%]. FIG. 2C shows left ventricular systolic function measured as fractional shortening (FS, WT=34%) between DCM+vehicle and DCM+DRI mice. FIG. 2E shows differences in cardiac output (CO, WT=15.5 mL/min) between DCM+vehicle and DCM+DRI mice. FIG. 2F shows Pearson's correlation analysis of 90-day EF vs. survival. FIG. 2G shows Pearson's correlation analysis of cardiac output (CO) vs. survival. DCM control mice (DCM+vehicle, solid circle, n=20), DCM mice treated with DRI (DCM+DRI, solid square, n=27). Differences between groups were analyzed by Mantel-Cox test and Mann-Whitney test. Pearson's correlation coefficient (rₚ) and p-values are shown. Data are represented as mean±SE, *p<0.05, **p<0.01 (DCM+vehicle vs. DCM+DRI).

FIG. 3 shows morphometric changes at 90 days. Heart weight to body weight (HW/BW, %) and lung weight to body weight (LW/BW, %) ratios at a 90-day collection of censored subgroups. DCM control (DCM+vehicle) and aliskiren treated (DCM+DRI) group sizes are shown. For reference, the mean ratios for wild-type mice (WT, n=4) for HW/BW are shown as a blue dashed line and the LW/BW as a solid blue solid line. Data were analyzed with two-tailed unpaired t-test and represented as mean±SE. Not significant (NS), ⁺⁺p<0.01, ⁺⁺⁺p<0.001, ⁺⁺⁺⁺p<0.0001 (hatch mark square WT vs. DCM+vehicle; solid square, WT vs. DCM+DRI), *p<0.05 (DCM+vehicle vs. DCM+DRI).

FIGS. 4A, 4B, 4C, 4D, 4E, 4F, 4G, 4H, and 4I show the effects of direct renin inhibition on systemic changes in mouse whole-body composition associated with HF progression. FIG. 4A shows age-related changes in body weight in DCM mice with (DCM+DRI) or without renin inhibition (DCM+vehicle) by comparison to wild-type (WT, without DCM) mice. FIG. 4B shows age-related changes in total water in DCM mice with (DCM+DRI) or without renin inhibition (DCM+vehicle) by comparison to wild-type (WT, without DCM) mice. FIG. 4C shows age-related changes in extracellular water (ECW) in DCM mice with (DCM+DR1) or without renin inhibition (DCM+vehicle) by comparison to wild-type (WT, without DCM) mice. FIG. 4D shows age-related changes in fat. FIG. 4E shows age-related changes in lean mass in DCM mice with (DCM+DRI) or without renin inhibition (DCM+vehicle) by comparison to wild-type (WT, without DCM) mice. FIG. 4F shows age related changes in combined fat and lean mass in DCM mice with (DCM+DRI) or without renin inhibition (DCM+vehicle) by comparison to wild-type (WT, without DCM) mice. WT (solid triangle; n=9-15), DCM+vehicle (solid circle; n=10-27), DCM+DRI (solid square; n=17-29). FIG. 4G shows Pearson's correlation between survival (days) and body weight measurements at 90 days. FIG. 4H shows Pearson's correlation between survival (days) and ECW measurements at 90 days. and FIG. 4I shows Pearson's correlation between survival (days) and fat measurements at 90 days. DCM+vehicle mice (n=20) and DCM+DRI mice (n=27). Differences between groups were analyzed by two-way ANOVA. Data are represented as the mean±SE. Pearson's correlation coefficient (rₚ) and p-values shown. ⁺p<0.05, ⁺⁺p<0.01, ⁺⁺⁺⁺p<0.0001 (WT vs. DCM+vehicle; WT vs. DCM+DRI), *p<0.05, ***p<0.001, ****p<0.0001 (DCM+vehicle vs. DCM+DRI).

FIGS. 5A, 5B, 5C, and 5D show cardiac-specific overexpression of corin affects heart function in both early and late phase post myocardial infarction (MI). FIG. 5A shows the dynamic changes in ejection fraction (EF %) (assessed by echocardiography), FIG. 5B shows the lung weight to body weight ratio (LW/BW %), FIG. 5C shows the systemic extracellular water (edema) assessed by quantitative magnetic resonance (QMR) and FIG. 5D shows heart weight to body weight ratio (HW/BW %) in mouse groups of WT vs. corin-Tg post-MI. Portions of data from FIGS. 5A, 5B and 5D (WT group at non-MI, 3 hrs, 24 hrs and 3 days) have been published previously and are included here to reduce animal numbers. Differences between WT and corin-Tg at each study time point were analyzed by 2way ANOVA with Sidak's multiple comparisons. Data represent means±SE of n=3-34 mice per group at each time point. *P<0.05, **P<0.01, ****P<0.0001 and not significant.

FIGS. 6A, 6B and 6C show cardiac corin-Tg(i) (catalytically inactive corin) overexpression reduces pleural effusion, edema, systemic water retention in female mice with DCM. FIG. 6A shows pleural effusions (PE) prevalence; bars represent percent affected mice analyzed by Fishers exact test and FIG. 6B shows lung weight to-body weight ratio (LW/BW) at 90 days of age, analyzed by one-way ANOVA with Newman-Keuls multiple comparison test. The number of mice per group is shown and the values for control mice without DCM (corin-WT/WT, wt,wt) are indicated by dotted lines (n=8). Age related changes in extracellular water (ECW) are shown in FIG. 6C. Experimental groups were: corin-Tg(i)/DCM (square, tg,tg, n=6-11), corin-WT/DCM (circle, wt,tg, n=20-25) and WT (dotted lines, n=15-18) mice. Data are represented as mean±SE; ****p<0.0001, **p<0.01 (tg,tg or wt,tg vs. wt,wt); ++++p<0.0001, ++p<0.01 (tg,tg vs. wt,tg).

FIGS. 7A, 7B, 7C, 7D, 7E, and 7F show that dietary sodium restriction suppresses pleural effusions, edema and prolongs survival in mice with DCM on a normal or low sodium (low) diet. FIG. 7A shows pleural effusions (PE) prevalence; bars represent percent of affected mice out of total mice in each group. PE was not detected in any WT controls. FIG. 7B shows Lung weight (LW). FIGS. 7C, 7D, and 7E show age-related changes corresponding to HF progression from C to D stages in extracellular water (ECW) (FIG. 7C), body weights (BW) (FIG. 7D), and total water (FIG. 7E). In FIGS. 7A and 7B Groups were 20 weeks of age; DCM mice on NSD n=14, on LSD n=7; WT mice (dashed line, n=9). In FIGS. 7C, 7D, and 7E: DCM mice enrollment n=9-11 per group; WT (dashed line, n=14-15 per group). FIG. 7F shows Kaplan-Meier survival curves of DCM mice receiving a low sodium diet (LSD) (n=32 deaths) vs. a normal sodium diet (NSD) (n=29 deaths); WT controls (dashed line, n=8-10). DCM mice on LSD (closed symbol) or NSD (open symbol). Data are presented as mean±SEM. ***p<0.001, *p<0.05 (DCM vs. WT) mice, ++p<0.01, +p<0.05 (DCM on NSD vs. DCM on LSD). Data were assessed by a Fisher's exact test (for FIG. 7A), by one-way ANOVA with Newman-Keuls multiple comparison test (for FIG. 7B), by two-way ANOVA with Bonferroni multiple comparison test (for FIGS. 7C, 7D, and 7E) or by the Kaplan-Meier method and the log-rank (Mantel-Cox) test (for FIG. 7F)

### DETAILED DESCRIPTION OF THE INVENTION

### Acronyms

ANP atrial natriuretic peptide

APRC: active plasma renin concentration

ARC: active renin concentration activity

BNP: B-type natriuretic peptide

cGMP: cyclic guanosine monophosphate

CO: cardiac output

DCM: dilated cardiomyopathy

DRI: direct renin inhibitor

ECW: extracellular water

EF: ejection fraction

HF: heart failure

HFrEF: heart failure reduced ejection fraction

HFpEF: heart failure preserved ejection fraction

NP: natriuretic peptide

PRAC: plasma renin activity concentration

PRR: pro-renin receptor

RAAS: renin-angiotensin-aldosterone-system

QMR: quantitative magnetic resonance

WT: wildtype

As used herein, the term “cardiovascular disease” refers to conditions of the heart including structural and functional abnormalities. Non-limiting examples comprise: heart failure (HF; a progressive heart disease that affects pumping action of the heart muscles as described herein); tachycardia (a heart rhythm disorder with heartbeats faster than usual, greater than 100 beats per minute in humans); cardiomyopathy (an acquired or inherited disease of the heart muscle which makes it difficult for the heart to pump blood to other parts of the body); coronary artery disease (a condition where the major blood vessels supplying the heart are narrowed); angina (chest discomfort or shortness of breath caused when heart muscles receive insufficient oxygen-rich blood); ventricular tachycardia (fast heart beat rhythm of the ventricles), myocardial infarction (death of heart muscle caused by a loss of blood supply); congenital heart defect (abnormality in the heart that develops before birth); atrial fibrillation (a disease of the heart characterized by irregular and often faster heartbeat); ventricular fibrillation (a serious heart rhythm problem in which the heart beats quickly and out of rhythm).

As used herein, “administering” and the like refer to the act physically delivering a composition or other therapy (e.g. a DRI, e.g., aliskiren) described herein into a subject by such routes as oral, mucosal, topical, transdermal, suppository, intravenous, parenteral, intraperitoneal, intramuscular, intralesional, intrathecal, intranasal or subcutaneous administration. Parenteral administration includes intravenous, intramuscular, intra-arterial, intradermal, subcutaneous, intraperitoneal, intraventricular, and intracranial administration. When a disease, disorder or condition, or a symptom thereof, is being treated, administration of the substance typically occurs after the onset of disease, disorder or condition or symptoms thereof. When a disease, disorder or condition, or symptoms thereof, are being prevented, administration of the substance typically occurs before the onset of the disease, disorder or condition or symptoms thereof.

As used herein, the terms “subject” and “patient” are used interchangeably. As used herein, a subject can be an animal (amphibian, reptile, avian, fish, or mammal) such as a non-primate (e.g., cows, pigs, horses, cats, dogs, rats, etc.) or a primate (e.g., monkey, ape and human). In specific embodiments, the subject is a human. In one embodiment, the subject is a mammal (e.g., a human) having a disease, disorder or condition described herein. In another embodiment, the subject is a mammal (e.g., a human) at risk of developing a disease, disorder or condition described herein. In certain instances, the term patient refers to a human under medical care or animals under veterinary care.

The terms “treating” or “treatment” refer to any indicia of success or amelioration of the progression, severity, and/or duration of a disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; or improving a patient's physical or mental well-being.

The term “effective amount” as used herein refers to the amount of a therapy or medication (e.g., DRI provided herein) which is sufficient to reduce and/or ameliorate the severity and/or duration of a given disease, disorder or condition and/or a symptom related thereto. This term also encompasses an amount necessary for the reduction or amelioration of the advancement or progression of a given disease (e.g., cardiovascular), disorder or condition, reduction or amelioration of the recurrence, development or onset of a given disease, disorder or condition, and/or to improve or enhance the prophylactic or therapeutic effect(s) of another therapy. In some embodiments, “effective amount” as used herein also refers to the amount of therapy provided herein to achieve a specified result.

As used herein, and unless otherwise specified, the term “therapeutically effective amount” of an DRI described herein is an amount sufficient enough to provide a therapeutic benefit in the treatment or management of a cardiovascular disease, or to delay or minimize one or more symptoms associated with the presence of the cardiovascular disease. A therapeutically effective amount of an agent (e.g., DRI) described herein, means an amount of therapeutic agent, alone or in combination with other therapies, which provides a therapeutic benefit in the treatment or management of the cardiovascular disease. The term “therapeutically effective amount” can encompass an amount that improves overall therapy, reduces or avoids symptoms or causes of cardiovascular disease, or enhances the therapeutic efficacy of another therapeutic agent.

A therapy is any protocol, method and/or agent that can be used in the prevention, management, treatment and/or amelioration of a given disease, disorder or condition. In certain embodiments, the terms “therapies” and “therapy” refer to a drug therapy, biological therapy, supportive therapy, radiation therapy, and/or other therapies useful in the prevention, management, treatment and/or amelioration of a given disease, disorder or condition known to one of skill in the art such as medical personnel.

This also accounts for any water-based fluids contained within compartments (e.g. urinary bladder, which can be negated if patient voids prior to QMR measurement).

As described herein, the term “edema” refers to excess extracellular water (ECW) or retention of ECW.

As described herein, the term “total water” refers to total body water that is the water content of a human or animal body that is contained in the tissues, the blood, the bones and elsewhere. The percentages of intracellular water and extracellular water add up to total body water (TBW).

As described herein, the term “sarcopenia” refers to degenerative loss of skeletal muscle mass, quality, and strength associated with aging and pathological conditions including advanced HF. Congestive HF can cause sarcopenia. Relating to cardiac sarcopenia, muscle mass loss is generally greater than expected for age and sex.

As described herein, the term “cachexia” refers to a complex syndrome associated with underlying illness causing ongoing combined muscle and fat mass loss that is not entirely reversed with nutritional therapy. A bodyweight loss of >5-6% characterizes cardiac cachexia.

Companion Diagnostic (CDx) assays, as defined by the FDA, are in vitro diagnostics (IVD) devices that provide information essential for the safe and effective use of a corresponding therapeutic product. The FDA specifies three main areas where a CDx assay is essential: 1) Identify patients who are most likely to benefit from a particular therapeutic product; 2) Identify patients likely to be at increased risk of serious adverse reactions as a result of treatment with a particular therapeutic product; and 3) To monitor response to treatment for the purpose of adjusting treatment (e.g., schedule, dose, discontinuation) and to achieve improved safety or effectiveness. A CDx can be used both to predict outcome (efficacy and safety) and to monitor response. The FDA has approved or cleared 35 CDx devices (as of 2018), which are available for the treatment of specific leukemias, gastrointestinal tumors, breast cancers, ovarian cancers, melanomas, lung cancers, and colorectal cancers, while no approved CDx assays are available for the treatment of heart failure related edema, muscle-wasting or sarcopenia or other complications.

As described herein, the term “asymptomatic” may refer to a subject without a condition as described herein. In another embodiment, the term “asymptomatic” may refer to a patient diagnosed with a condition (e.g. heart failure) but currently has no symptoms of the condition. In further embodiments, the term “asymptomatic” may refer to a patient diagnosed with a condition (e.g. heart failure) but has responded to previous therapy and displays no symptoms of said condition.

This claim is only to patients with diagnosed heart failure—with symptoms or without symptoms. Asymptomatic means the patient has responded to previous therapy (Ex: furosemide for PE during an acute condition). Patients suspected to have HF would be those ‘at risk’ (family/genetics; comorbidities; drug treatments—example doxorubicin or other chemotherapeutics). ECW can start to increase prior to clinical signs associated with fluid retention assessed by physicians.

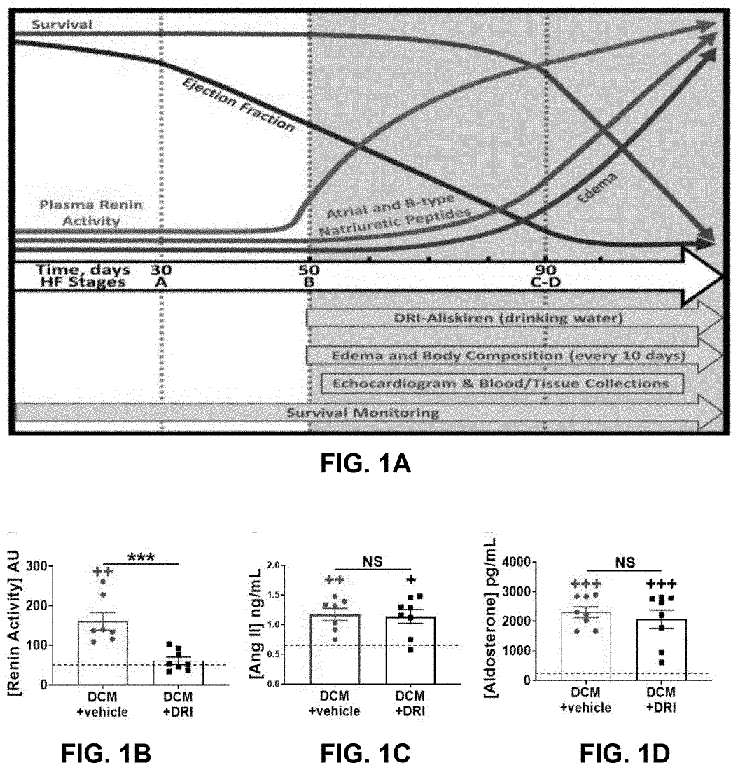

Referring now to FIG. 1A-7F, the present invention features a new method for personalized treatment for conditions that cause ECW retention/edema, lean muscle mass loss, and/or fat loss using QMR for diagnostic, prognostic, treatment tailoring and monitoring purposes of HF and HF-related conditions. This technology allows for a method to measure body fluid dynamics and body mass compositions for prognosis, treatment tailoring and monitoring strategies (e.g., treatment management) for disease conditions that cause ECW retention, lean muscle mass loss, and/or fat loss. Body fluid dynamics can be reflected by ECW content and edema and body compositions may comprise lean muscle mass and fat mass. In some embodiments, the present invention uses biomarkers such as water content, lean muscle mass, and fat content to use the changes in these biomarkers as indicators for HF prognosis, disease progression, and personalized treatment and monitoring strategies. The ability to assess accumulation of edema using QMR allows for medical provider-independent assessments.

The present invention features a method for treating a patient who is suffering from a heart condition. In some embodiments, the method comprises determining whether the patient has extracellular water (ECW) retention by 1. performing quantitative magnetic resonance (QMR) to measure extracellular water content of said patient and 2. determining a status of the heart condition in said patient or risk of death of said patient based on measured ECW content. In some embodiments, if the patient has an increase of extracellular water retention of more than 5% compared to a baseline level, a treatment is administered to the patient.

The present invention may also feature a method of monitoring effectiveness of a treatment in a patient with a heart condition. In some embodiments, the method comprises determining whether the patient has extracellular water (ECW) retention by 1. performing quantitative magnetic resonance (QMR) to measure extracellular water content of said patient and 2. determining a status of the heart condition in said patient or risk of death of said patient based on measured ECW content. In other embodiments, the method comprises monitoring the effectiveness of the treatment in the patient with a heart condition by determining ECW retention changes compared to a baseline level. In some embodiments, if the patient has an increase of extracellular water retention of more than 5% compared to a baseline level, a different treatment is administered to the patient. In some embodiments, if the patient has a decrease of extracellular water retention more than 5% compared to a baseline level, the patient maintains the same treatment.

In some embodiments, the heart condition comprises heart failure (HF), HF-associated and non-associated -sarcopenia/cachexia, -necrosis, -liver disease, and/or -kidney disease, HF-reduced ejection fraction (HFrEF), HF-preserved ejection fraction (HFpEF), heart dysfunction.

In some embodiments, if the patient has an increase of ECW retention of more than about 5% compared to a baseline level, a treatment is administered to the patient. In some embodiments, if the patient has an increase of ECW retention of more than about 6% compared to a baseline level, a treatment is administered to the patient. In some embodiments, if the patient has an increase of ECW retention of more than about 7% compared to a baseline level, a treatment is administered to the patient. In some embodiments, if the patient has an increase of ECW retention of more than about 8% compared to a baseline level, a treatment is administered to the patient. In some embodiments, if the patient has an increase of ECW retention of more than about 9% compared to a baseline level, a treatment is administered to the patient. In some embodiments, if the patient has an increase of ECW retention of more than about 10% compared to a baseline level, a treatment is administered to the patient. In some embodiments, if the patient has an increase of ECW retention of more than about 12% compared to a baseline level, a treatment is administered to the patient. In some embodiments, if the patient has an increase of ECW retention of more than about 15% compared to a baseline level, a treatment is administered to the patient.

In some embodiments, a ECW retention of more than 20% above baseline indicates a pathological value. In some embodiments, a ECW retention of greater than 20% above baseline in combination with other diagnostic tools may be used to diagnose a patient with a condition described herein.

In some embodiments, if the patient has a decrease of ECW retention of about 5% compared to a baseline level, a treatment is administered to the patient. In some embodiments, if the patient has a decrease of ECW retention of about 6% compared to a baseline level, a treatment is administered to the patient. In some embodiments, if the patient has a decrease of ECW retention of about 7% compared to a baseline level, a treatment is administered to the patient. In some embodiments, if the patient has a decrease of ECW retention of about 8% compared to a baseline level, a treatment is administered to the patient. In some embodiments, if the patient has a decrease of ECW retention of about 9% compared to a baseline level, a treatment is administered to the patient. In some embodiments, if the patient has a decrease of ECW retention of about 10% compared to a baseline level, a treatment is administered to the patient. In some embodiments, if the patient has a decrease of ECW retention of about 12% compared to a baseline level, a treatment is administered to the patient. In some embodiments, if the patient has a decrease of ECW retention of about 15% compared to a baseline level, a treatment is administered to the patient.

In some embodiments, a treatment is administered to a patient until ECW retention levels reach normalization. As used herein, “normalization” refers to healthy/non-diseased age and sex matched populations range (median+/−SD).

In other embodiments, the treatment administered to the patient is the same treatment, at a higher lower dose.

In some embodiments, the treatment administered to the patient is a different treatment. In other embodiments, the different treatment administered to the patient is the same treatment, at a higher dose. In some embodiments, the patient is administered the same treatment in combination with another treatment. In other embodiments, the treatment administered to the patient is the same treatment, at a higher lower dose.

In some embodiments, the patient has no increase of ECW retention compared to a baseline level then the treatment administered to the patient is the same treatment.

Clinicians are restricted to ACC/AHA Guidelines, hospital protocols, and general best practices for administering or prescribing maximum therapeutic doses. Without wishing to limit the present invention to any theories or mechanisms it is believed that heart failure patients may develop a tolerance or lack of effect to a particular therapy (e.g., ACE inhibitors) overtime. Changes in medications or treatment strategies should be initiated when there is a lack of response to pathological values of ECW (>20% normal) despite having reached maximum therapeutic dose(s). When this occurs, clinicians should pivot therapy to a different class of drug either independently or synergistically with the original therapy. The goal is to normalize ECW levels and titrate therapeutic doses to the lowest effective dose in order to minimize side effects. Patients can be measured longitudinally to adjust therapies on an as needed basis, resulting in a personalized medicine approach to heart failure treatment.

Pre-clinical and clinical studies also can be improved using edema and body composition markers (fat, lean muscle) in order to objectively measure changes in fluid and mass dynamics longitudinally. This technology addresses the technical problem by improving precision and effectiveness of HF and HF -associated and non-associated disease detection and treatment.

Non-limiting examples of the advantages of this technology comprise: 1) medical provider-independent non-invasive assessment of sodium and water retention; 2) assessment of HF progression and prognosis; 3) access edema in lungs and other locations; 4) personalize therapy to improve quality and prolong life; and 5) diagnose, monitor and treat sarcopenia/cachexia associated with HF and HF-non-associated edema/sarcopenia/cachexia, kidney disease, liver disease and muscular dystrophy.

In some embodiments, changes in body fluid dynamics comprise ECW retention and the final outcome edema. Non-limiting examples of changes in body composition comprise changes in lean muscle mass and fat content. Edema is the abnormal accumulation of fluid in certain tissues within the body. The accumulation of fluid may be under the skin, usually in dependent areas such as the legs (peripheral edema, or ankle edema), or it may accumulate in the lungs (pulmonary edema), or the abdomen/peritoneum (ascites). The location of edema can provide the health care practitioner the first clues in regard to the underlying cause of the fluid accumulation. This late identification of underlying pathology/disease development/progression is the advantage of initiating QMR in surveying and monitoring HF and HF-associated diseases with high potential for these body compositional changes.

In appropriate circumstances, the condition that may in part cause or caused by ECW retention/edema, lean muscle mass loss/sarcopenia, and/or fat loss/cachexia comprises HF, HFrEF, HF with preserved ejection fraction (HFpEF), heart dysfunction, HF-associated and non-associated -sarcopenia/cachexia, -necrosis, -liver disease, -kidney disease, -muscular dystrophy, and/or any condition or treatment that causes ECW retention/edema, lean muscle mass loss/sarcopenia, and/or fat loss/cachexia. In preferred embodiments, HF comprises HF-associated and non-associated -sarcopenia/cachexia, -necrosis, -liver disease, and/or -kidney disease, HFrEF, HFpEF, heart dysfunction, ascites, organ hypoperfusion, and/or shock.

In some embodiments, the present invention may be used in combination with other diagnostic methods to accurately diagnose a patient with a condition that causes or is caused by ECW. In some embodiments, extracellular water content; total water content; muscle/lean mass; and/or fat mass are biomarkers that may be used in combination with Echocardiography, cMRI, and other clinical factors to help confirm the Stage of HF. In other embodiments, ECW helps determine the stage of progression for a particular condition described herein.

The present invention further features changes in fluid dynamics (e.g., ECW retention, edema) and body compositions (e.g., lean muscle mass, fat mass) are detected longitudinally and longitudinal levels can be compared to baseline levels. In some embodiments, QMR is performed in asymptomatic individuals, wherein asymptomatic individuals are individuals without the condition. In appropriate circumstances, baseline levels are normal levels from an aggregate population of asymptomatic individuals without the condition. In other circumstances, baseline levels are relative baseline levels for the individual patient at the time of initial presentation or diagnosis of the condition.

In preferred embodiments, QMR is performed at various times, longitudinally, throughout progression of the condition. Non-limiting examples of when QMR is performed comprise: 1) at time of diagnosis or initial presentation of symptoms; 2) 12 hours post-diagnosis; 3) two to seven days post-diagnosis, 4) one month post-diagnosis, 5) three months post-diagnosis, or 6) >three months post-diagnosis.

In some embodiments, the change in ECW level is 10% to 20% of baseline, 20% to 50% of baseline, or >50% of baseline. In some embodiments, the change in lean muscle mass level is, 10% to 20% of baseline, 20% to 50% of baseline, or >50% of baseline. In some embodiments, the change in fat level is 10% to 20% of baseline, 20% to 50% of baseline, or >50% of baseline.

In some embodiments, the therapeutically effective drugs for heart dysfunction and HF comprise inhibitors to the renin pathway, which result in an alteration of renin activity levels. In other embodiments, the therapeutically effective drugs comprise diuretics (e.g., carbonic anhydrase inhibitors), dobutamine, therapeutics against angiotensin, aldosterone or neprilysin, beta blockers, antiarrhythmic agents, anticoagulants, cholesterol-lowering drugs (e.g., statins), and digoxin. Physically draining a cavity, and lifestyle changes (diet/nutritional alterations, exercise, stop smoking, etc) are also important interventional strategies in HF. In other embodiments, treatment comprises devices that improve or stabilize cardiac function comprising pacemakers, defibrillators, circulatory assistance, artificial hearts, transplantation.

In some embodiments, the therapeutically effective drugs for heart dysfunction and HF comprise diuretics (e.g. chlorothiazide, HCTZ lasix), beta blockers, Angiotensin converting enzyme inhibitors (ACE inhibitors), Angiotensin II receptor blockers (ARBs), Angiotensin Receptor-Neprilysin Inhibitors (ARNi), Mineralocorticoid Receptor Antagonists (MRA), direct renin inhibitors (DRI), or Sodium-glucose co-transporter-2 (SGLT2) inhibitors.

In other embodiments, the methods of the present invention personalize initiation and continuation of therapy based on personal clinicopathologic characteristics specific to the patient. Non-limiting examples of personal clinicopathologic characteristics comprise measured extracellular water content, total water content, muscle/lean mass, fat mass, pro-renin activity, (pro)-renin receptor levels, plasma renin activity assayed and defined as PRA, active renin concentration, active plasma renin concentration, and/or plasma renin activity concentration or other methods, and/or disease etiology comprising heart rate, heart rhythm, genetic causes, sex, race, age, geographic location, diet, exercise habits, smoking status and/or heart dysfunction specific to the patient.

In some embodiments, the present invention features a method for prolonging life, for personalizing initiation and continuation of therapy, for determining the onset of the condition (e.g., HF, liver disease, kidney disease). Non-limiting examples of prolonging life comprise prolonging life by at least 1 month, at least 3 months, at least 6 months, or at least 1 year or greater. In other embodiments, the methods reduce progression of the condition or heart dysfunction, diminishes HF or risk of HF, delay the transition from heart dysfunction to clinical HF, and/or determine the onset of the condition or HF

In appropriate circumstances, the method provides for an objective, medical provider-independent assessment of HF stage transitions (e.g., objective measure of transition between HF stages B and C). Additional embodiments feature a method for medical provider-independent assessment of sodium and water retention/edema and sarcopenia/cachexia.

Other embodiments of the present invention may feature a method for assessing new therapeutic approaches for ECW content retention/edema and/or lean muscle mass losslsarcopenia, and/or fat loss/cachexia and may be used as a Companion Diagnostic for said condition or HF. In other embodiments, the methods of the present invention are used to stratify patients with HF or those at risk for HF to determine appropriate medication and level of medication and/or intervention(s) to treat further progression of HF. In some embodiments, the methods of the present invention use changes in fluid dynamics (e.g., ECW, total water) and body compositions (e.g., lean muscle mass, fat mass) for a longitudinal assessment of HF prognosis and progression. The methods of the present invention also can longitudinally assess at risk and/or HF prognosis and progression.

### EXAMPLES

The following are non-limiting examples of practicing the present invention. It is to be understood that the invention is not limited to the examples described herein. Equivalents or substitutes are within the scope of the invention.

### Methods

Mice: Dilated cardiomyopathy (DCM) is a major cause of HF, which is associated with pathological dilation of the heart ventricles and declines in heart contractile or systolic function. A well-established, translational mouse model of DCM with reduced ejection fraction (rEF) fulfills the criteria of the American Heart Association Scientific Statement for Animal Models of Heart Failure was utilized in the examples below. The mice used for this study were randomly assigned female littermates with or without (DCM on a C57BL/6 background. DCM mice express a transgene dominant-negative CREB transcription factor specific to cardiomyocytes and consistently develop the progressive stages (A-D) of HF similar to those described for humans. In female mice with DCM renal function remains within a normal range up to the terminal HF stage, as measured by plasma BUN and creatinine. DCM+vehicle (n=28) and DCM+DRI (n=29) were compared to each other. A group of congenic WT mice (n=20) was used for reference. In sub-groups of 90-day old mice (n=8/group), terminal blood was collected via cardiocentesis with ethylenediaminetetraacetic acid (EDTA)-aprotinin syringes to prevent proteolysis of targeted proteins and dissected organs were weighed. The blood samples were centrifuged at 3000 rpm for 20 min at 4° C. Plasma samples were aliquoted and stored at −80° C. until analysis. All analysis and health/death reports were recorded by investigators and animal facility technicians who were blinded to the mouse genotype.

Direct Renin-Inhibitor Treatment: The direct renin-inhibitor group (DCM+DRI) was administered aliskiren hemifumarate (BOC Sciences, Shirley, N.Y., USA) at 100 mg/kg/day orally via single source drinking-water in autoclaved hanging bottles. Dose calculations were based on an average consumption of 5 mL of water/day/mouse. The bioavailability of aliskiren is low in humans and only 2.6% orally in rats. Previous studies administering 15-50 mg/kg/day via subcutaneous osmotic pumps showed no alteration in blood pressure. The oral dose was chosen to closely mimic the human route of delivery, while not altering the blood pressure, associated with elevated plasma Ang II-aldosterone levels, given the low bioavailability of the drug in rodents. To prepare the solution, aliskiren powder was dissolved by shaking in volume measured autoclaved 3 ppm hyperchlorinated facility water, mixed fresh every 72 h and shaken daily to resuspend. No issues with palatability or clinical dehydration were noted throughout the study as assessed by blinded husbandry and veterinary staff. There were no side effects or abnormal clinical observations in our mice throughout the treatment period. The aliskiren in drinking water was well tolerated and consumed at expected levels for plain water. The administration was started at 50 days of age to coincide with the previously identified timeline of increased renin activity and development of stage B HF in female DCM mice]. Aliskiren water was provided ad libitum throughout life. All untreated mice (WT and DCM+vehicle) received ad lib 3 ppm hyperchlorinated facility water via an automated watering system (Edstrom, Waterford, Wis., USA).

Body Composition: Body composition was objectively quantified by a single-blinded and experienced operator. Bodyweight, free water (systemic extracellular water, ECW), total water, body fat, and lean mass were recorded longitudinally. Mice were measured every tenth day between 50-100 days using quantitative magnetic resonance (QMR) technology with an EchoMRI™ 4-in-1 Analyzer (Echo Medical Systems, Houston, Tex., USA). The machine was calibrated daily per standard operating procedure using the provided canola oil (54.3 g) phantom. Briefly, mice were weighed (Scout Pro SP401, Ohaus Corporation, Pine Brook, N.J., USA) and loaded into a tube restrainer specific to the system. Mice were fully conscious and minimally restrained throughout each 60-90 s recording and were returned to their home enclosure following measurement.

Echocardiography: The standard transthoracic exam was performed using a Vevo 2100 Imaging System (VisualSonics; Toronto, ON, Canada) with a 30 MHz transducer (MS 400) as previously reported. All imaging was performed at 90 days of age (mice were treated for 35 days before subjecting to echocardiogram). Briefly, mice were induced with 3-5% isoflurane in oxygen and fur removed with depilatory cream (Nair, Church & Dwight Co. Inc., Princeton, N.J., USA). Maintenance anesthesia was held at 2% isoflurane in oxygen throughout the two-dimensional and M-mode recordings of the left ventricle (LV) in parasternal long-axis, short-axis, and four-chamber views. Mouse physiology was maintained at an anesthetized heart rate of 450±50 beats per minute and 37±1° C. rectal temperature. The analysis was blindly completed post-recording using Vevo LAB software (3.1.0, VisualSonics) with three cardiac cycles traced to produce mean values. Ejection fraction (EF, %), fractional shortening (FS, %), LV mass corrected (LVMc, mg), and cardiac output (CO, mL/min) were calculated using standard equations within the software.

Enzyme Immunoassay: Plasma angiotensin II (Ang II), aldosterone, atrial natriuretic peptide (N terminus-ANP), cyclic guanosine monophosphate (cGMP), neprilysin, and corin levels were measured by enzyme immunoassays according to the manufacturers' protocols (Phoenix Pharm. Inc., Burlingame, Calif., USA; Abcam Inc., Cambridge, Mass., USA; Enzo Life Sciences Inc., Farmingdale, N.Y., USA; Boster Biological Technology, Pleasanton, Calif., USA; USCN Life Science Inc., Houston, Tex., USA) as previously reported.

Plasma Renin Activity Assay: Renin enzymatic activity from EDTA-aprotinin supplemented mouse plasma samples (described in the sub-section 4.2) were measured in a 96-well microplate (Synergy HT reader and Gen5 v1.09 software, BioTek Instruments, Inc., Winooski, Vt., USA) and quantified using exogenous fluorescence resonance transfer (FRET) peptide substrates of renin FRET-QXL™ 520/5-FAM, optimized for mouse renin (SensoLyte 520 mouse renin assay kit, AnaSpec, Fremont, Calif., USA) as previously reported. Cleavage of the FRET substrate by mouse renin results in the recovery of quenched fluorescence of 5-FAM, which was detected at excitation/emission=490/520 nm with minimum autofluorescence of plasma samples. The 5-FAM fluorescent reference standard curve was used for results quantification. It is important to note that the plasma renin activity concentration assay differs from plasma renin activity and active renin concentration (ARC)/active plasma renin concentration (APRC) which have been historically used to report active renin in clinical trials.

Statistical Analysis: Statistical analyses were performed with GraphPad Prism 7.04 software (GraphPad Software, La Jolla, Calif., USA) using Student's t-test, one-way ANOVA, or two-way ANOVA with Tukey's multiple comparisons test (unless otherwise indicated) and Pearson's correlation. Survival was analyzed using Kaplan-Meier curves with the Mantel-Cox test. Differences were considered significant if p≤0.05. The number of animals (n) is indicated in the figures or figure legends. Data are represented as mean±SE.

### Example 1

### Stages and Characteristics of Heart Failure Development in DCM-HFrEF Mouse Model and Suppression of Elevated Plasma Renin Activity in Females with Dilated Cardiomyopathy (DCM)

FIG. 1A shows HF development from Stage A (no HF), to Stage B (structural heart disease), through Stage C (edema, symptoms), Stage D (severe HF) and death. Female mice with DCM begin to show declines in heart systolic function (ejection fraction; EF) and increases in plasma renin activity around 7 weeks of age (Stage B HF), which is prior to the development of progressive edema, further declines in systolic function, rises in atrial/B-type natriuretic peptide (ANP/BNP) and death (FIG. 1A). Female littermate mice with DCM were randomly assigned to receive no treatment (control) or the direct renin inhibitor (+DRI) aliskiren. Treatment with the DRI significantly reduced elevated plasma renin activity to normal levels as expected (P<0.01, FIG. 1B). Pathologically elevated plasma aldosterone levels were not modulated by treatment (FIG. 1C). The aldosterone to renin ratio was significantly increased in +DRI mice vs. controls (P<0.05, FIG. 1D).

### Example 2

### Renin Activity Suppression Prolongs Survival and Delays Progression of Left Ventricular Systolic Dysfunction

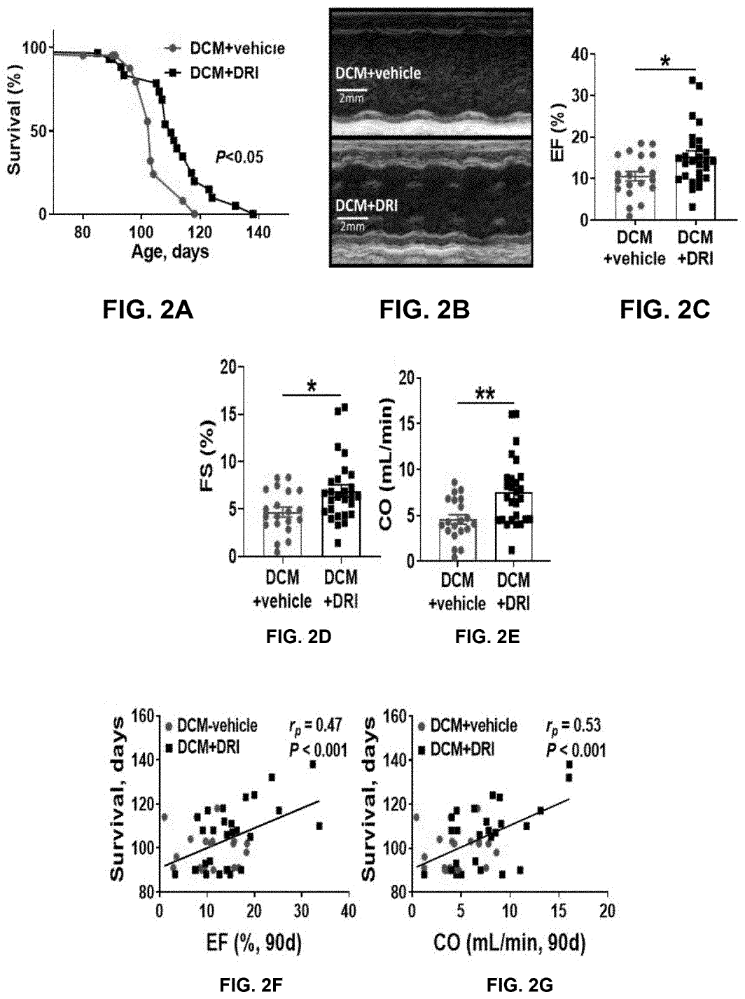

The effect of renin activity suppression was assessed in female mice with DCM as they pass progressively through the stages of HF development from Stage A (no HF), to Stage B (structural heart disease), through Stage C (edema, symptoms), Stage D (severe HF) and death. Three groups of female littermates were examined—DCM control, DCM+DRI and WT mice. WT littermates had 100% survival throughout the 140 day study (data not presented). +DRI mice outlived the control mice by 7% (median survival—110 vs. 103 days respectively, P<0.05, FIG. 2A). In the same experimental groups, cardiac structure and function were assessed by echocardiography at 90 days (Stage C HF with respect to control group). Systolic function in control mice was improved with DRI treatment as measured by ejection fraction (EF %, P<0.05, FIG. 2B) and fractional shortening (FS %, P<0.05, FIG. 2F). Cardiac output (CO; mL/min) was also improved with DRI treatment (P<0.01, FIG. 2C), reflecting changes in both heart rate (control 419±10 bpm vs. +DRI 469±14 bpm, P<0.01) and changes in stroke volume (control 11±1 μL vs. +DRI 16±1 μL, P<0.05). Contractile function assessed at 90 days by EF (rₚ=0.47, P<0.001, FIG. 2D) and CO (rₚ=0.53, P<0.05, FIG. 2E) were positively correlated with survival outcome.

### Example 3

### Normalization of Plasma Renin Activity Delays Development of Systemic Edema and Cachexia/Sarcopenia

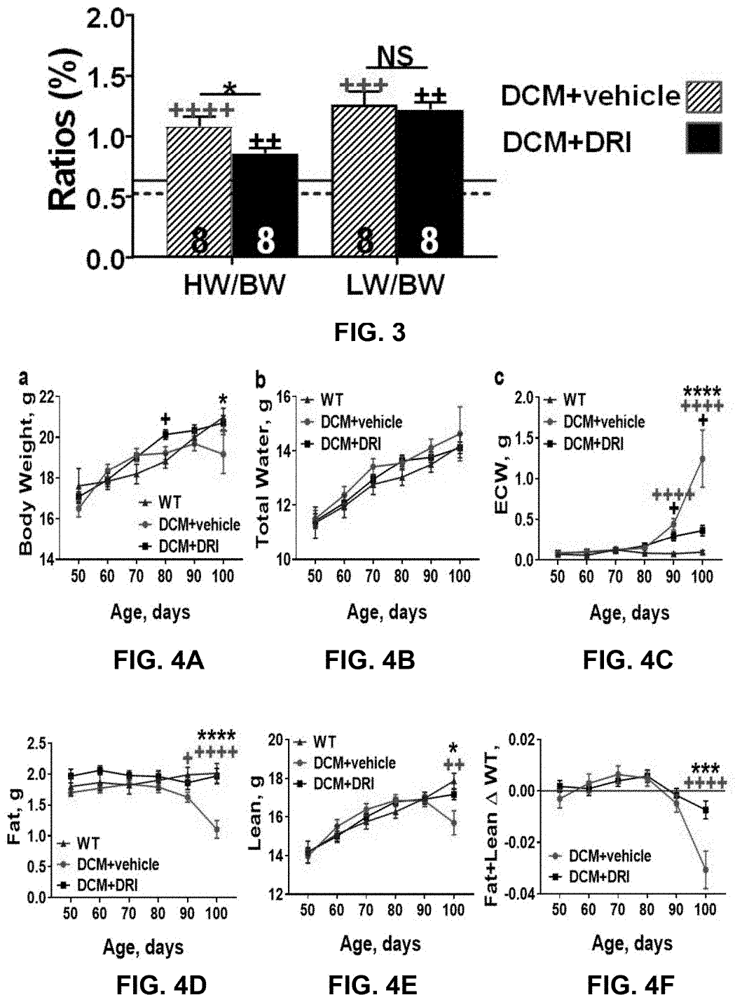

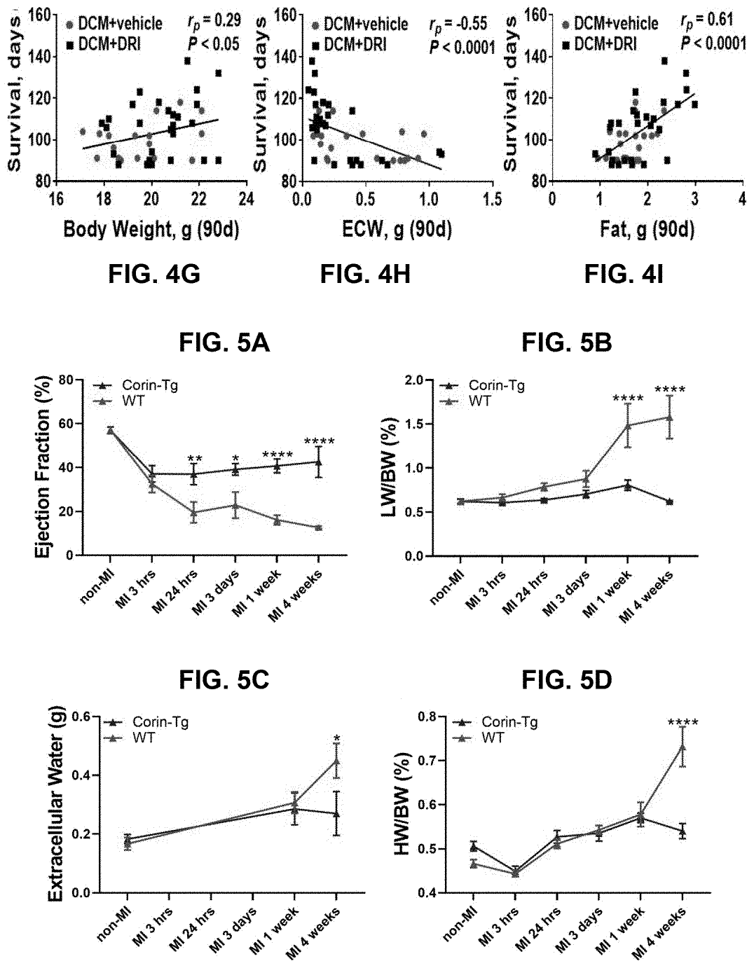

To evaluate the effects of renin activity normalization, mouse hearts and lungs were examined at 90 days of age. Control (DCM+vehicle) mice had a significant increase in heart to body weight ratios (HW/BW) compared to WT (p<0.0001, FIG. 3) and DCM+DRI (p<0.05, FIG. 3 mice. DRI-treated mice had an increased HW/BW ratio compared to WT (p<0.01, FIG. 3). DRI treatment reduced the gross increase in cardiac weight of control (DCM+vehicle) mice by 20.4% (p<0.05). Both DCM+vehicle and DCM+DRI groups were characterized by an increased lung weight to body weight (LW/BW) ratio compared to WT (p<0.001 and p<0.01 respectively, FIG. 3). Body weights at 90 days were not significantly different between groups (FIG. 4A). As a result, the ratio differences in HW/BW and LW/BW appeared to be due to the increased cardiac and lung weights of the vehicle and DCM+DRI mice.

Similar to humans with DCM, mice with DCM develop heart dilation, which progresses to symptomatic HF, water retention, and edema. Edema is characterized not only by water retention in lung tissue (pulmonary and pleural), but also by systemic water accumulation in the cavities and/or tissues of the body (ascites and peripheral tissues).

To quantify systemic water retention, body composition was dynamically monitored in all experimental groups every 10 days starting from 50 days of age, using quantitative magnetic resonance (QMR). Body weights were relatively consistent until 100 days when weights appeared to decline in DCM+vehicle controls compared to WT and DCM+DRI mice (p<0.05 and p<0.05, FIG. 4A). Feed was provided to all mice ad-lib throughout the study; however, diet consumption was not specifically monitored. Total water measurements were similar among all groups throughout the study (FIG. 4B). Systemic extracellular water (ECW, reported as free water in QMR measurements) began to rise after 80 days and became significantly elevated at 90 days of age in DCM+vehicle mice (p<0.0001, FIG. 4C). The progressive increase of ECW continued at 100 days in control (DCM+vehicle) mice compared to WT (p<0.0001). Systemic ECW levels were significantly higher in DCM+vehicle vs. DCM+DRI mice when recorded at 100 days of age (p<0.0001, FIG. 4C).

The diagnosis of cardiac cachexia/sarcopenia in HF patients correlates strongly with an overall poor prognosis. Similarly, the DCM mice developed measurable wasting as they progressed from late Stage C to terminal Stage D HF (FIGS. 4D-4F). Body fat changed in the opposite pattern; treatment with DRI resulted in the maintenance of fat levels similar to WT through 100 days, whereas DCM+vehicle mice showed major fat losses by 100 days (p<0.0001, FIG. 4D). There was a significant decline in lean body mass in DCM control mice at 100 days, consistent with sarcopenia, by comparison to DCM+DRI and WT mice (FIG. 4E). Overall there was significant wasting (FIG. 4F) or cachexia, as measured by combined losses of fat and lean mass in DCM control mice by 100 days relative to WT (p<0.0001) and DCM+DRI mice (p<0.001).

Bodyweight at 90 days of age was marginally associated with survival (FIG. 4G). A better measure was systemic ECW, which was negatively correlated with survival outcomes (rₚ=−0.55, p<0.0001, FIG. 4H). Fat measurements had the best correlation (rₚ=0.61, p<0.0001, FIG. 4I) with survival. Lean changes (rₚ=0.17, p=0.2591) were not significantly correlated with survival.

The experiments described above were performed in a well-characterized preclinical mouse model of DCM that is translationally-relevant to human HFrEF. Mice with DCM pass through all stages of HFrEF (Stages A-D) with well-defined biomarker profiles, preservation of kidney function, and normal blood pressure through Stage D and death. This animal model allowed for the investigation of DRI-aliskiren in DCM without clinical confounders (e.g., nutritional, environmental, concurrent or underlying disease status, and coadministration of additional medications). Female DCM mice were randomized to begin treatment at 50 days when pathologically elevated plasma renin activity concentration is first detected and there is an initial decline in EF corresponding to Stage B HF (FIG. 1A) without the onset of edema and elevation of ANP/BNP plasma biomarkers. DCM+DRI mice showed delayed development of Stage C HF as evidenced by the reduced ECW accumulation (edema) and a 7% increase in overall survival, which is potentially equivalent to an additional 5.6 human years assuming an 80-year lifespan. The enhanced survival was associated with 1) improvement in LV CO, 2) a robust reduction in systemic edema, and 3) prolonged maintenance of normal overall body composition (bodyweight, fat, and lean mass). When compared to DCM+vehicle treated animals, the DCM+DRI animals showed a significant absolute increase in EF of 5% and a relative increase of 33%. Clinical studies have shown that in patients with low EF there is a direct correlation of EF with mortality, with higher EF being linked to better survival. One of the factors most correlated with mortality was ECW retention, which is linked to the onset of edema. It is interesting to note that ECW levels greater than one gram in both groups were associated with mortality, suggesting that these levels are not compatible with survival. Clinical studies investigating edema as a biomarker for mortality support these findings; however, routine monitoring of HF patients currently does not include the non-invasive quantification of ECW, which precedes the onset of clinically detectable and symptomatic edema].

In HF, cardiac cachexia is associated with increased rates of morbidity and mortality, independent of ventricular function and clinical symptoms. Most experts agree that bodyweight loss of >5-6% should be used to characterize cardiac cachexia. Serial QMR measurements permitted the detection of body fat and muscle losses, which may have been masked by simply following body weights as currently recommended for management of HF patients. Changes in body composition started at 90 days of age and became significant at 100 days with declines in body weight (−8.6% compared to WT), fat composition (−45%), and lean mass (−12%) evident in the DCM+vehicle control group. Whether these changes in body composition are due to changes in metabolism or appetite is unknown, as caloric intake was not measured. The role of RAAS modulation in the alteration of sarcopenia is recognized. The normalization of plasma renin activity attenuated cachexia and sarcopenia, independently of alteration of plasma Ang II levels, suggests that renin activity contributes directly or indirectly to this process.

Although there are important differences between patients, nearly everyone with HFrEF is currently treated with the same medications. The data presented herein show that normalizing the increased PRAC in experimental HFrEF significantly improved systolic function, delayed the onset of edema, diminished the development of cachexia/sarcopenia, and significantly extended survival. The data presented herein support the basis of present invention that increased plasma renin activity has deleterious effects in HF and indicates that, in appropriately identified individuals, the normalization of plasma renin activity may have protective effects by delaying the transition from early, asymptomatic to more severe and fatal HF. Overall, these preclinical data provide the first direct evidence that elevated renin activity contributes to the progression of dilated cardiomyopathy, systemic edema, cachexia/sarcopenia and mortality.

### Example 4

### Cardiac-Specific Overexpression of Corin Significantly Improves Heart Function and Delays Heart Failure Development Post Myocardial infarction (MI)

Cardiac function by echocardiography was evaluated at both early phase (3 hrs, 24 hrs or 3 days) and late phase (1 and 4 weeks) post-MI in WT-MI and corin-Tg-MI groups. Although ejection fraction (EF) dropped in both groups post-MI when compared to non-MI controls, corin-Tg-MI had better EF at most study time points (p<0.05, p<0.05, p<0.001 and p<0.01 respectively at 24 hrs, 3 days, 1 week and 4 weeks) in contrast to WT-MI groups (FIG. 5A). A similar trend was also confirmed by fractional shortening (data not shown). Pulmonary congestion, an important clinical sign of heart dysfunction, was more severe in WT-MI groups than in corin-Tg-MI groups evidenced by higher lung weight to body weight ratio (LW/BW, FIG. 5B) and systemic extracellular water (edema) assessed by quantitative magnetic resonance (QMR) (FIG. 5C). The patterns of LW/BW and systemic extracellular water increases in WT mice supports that pulmonary edema develops prior to pleural effusion in this translational model. There were no significant changes in HW/BW at early time points and only increased at 4 weeks post-MI (p<0.0001, FIG. 5D).

### Example 5

### Corin-Tg(i) Overexpression Reduces Pleural Effusion, Lung Edema and Systemic Water Retention in Mice with DCM

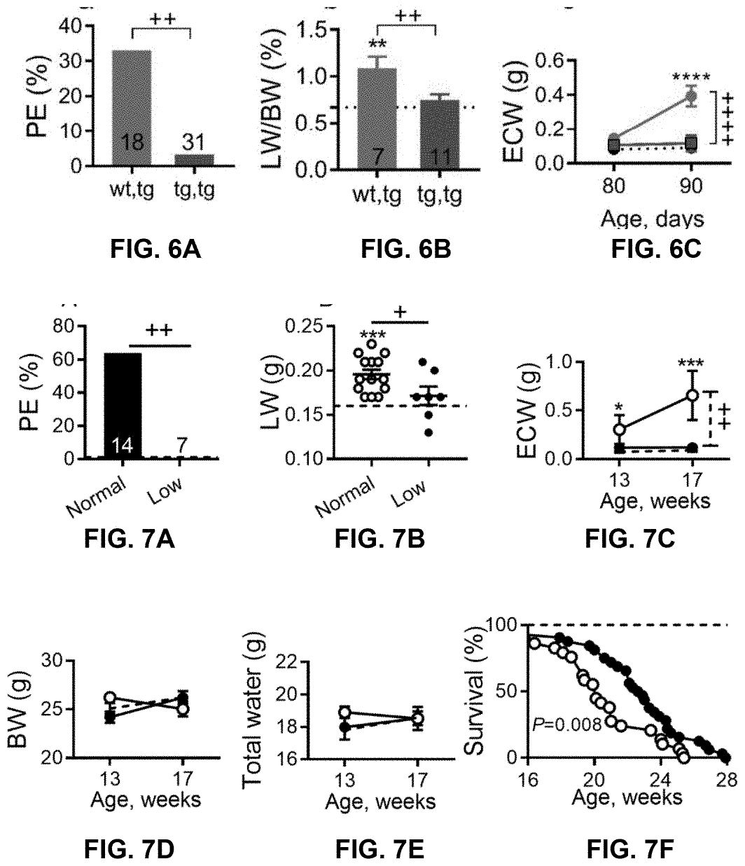

Pleural effusion, lung water retention or lung edema are clinical manifestations of advanced HF (Stages C-D HF) in humans and in DCM mice. Necropsy analysis of sub-groups of mice at 90 days of age confirmed the presence of pleural effusion and lung edema in corin-WT/DCM mice (FIGS. 6A and 6B). Pleural effusion was evident by presence of the pleural fluid in the thoracic cavity and lung edema was assessed by lung weight to body weight ratio (LW/BW, %). Pleural effusion prevalence was significantly decreased in corin-Tg(i)/DCM vs. corin-WT/DCM mice (FIG. 6A, 3.3 vs. 33%, p<0.01). Similarly, lung edema (LW/BW) was significantly reduced in corin-Tg(i)/DCM vs. corin-WT/DCM mice (FIG. 6B, p<0.05) but was not significantly different from WT group (FIG. 6B). Female DCM mice at Stages C-D HF, like humans, accumulate edema in the lungs, and peripheral tissues (systemic edema), which can be measured as increases in extracellular water (ECW; FIG. 6C) using noninvasive quantitative magnetic resonance (QMR) for body composition monitoring. By 90 days of age, corin-WT/DCM mice accumulated significantly elevated ECW levels as compared with corin-Tg(i)/DCM littermate mice (p<0.0001, FIG. 6C), though body weights were relatively comparable. Moreover, corin-Tg(i)/DCM mice maintained normal ECW levels, which were comparable with WT mice (FIG. 6C).

### Example 6

### Dietary Sodium Restriction Suppresses Development of Pleural Effusion, Systemic Edema and Prolongs Survival

The impacts of LSD on progression of DCM and HF were examined in male littermate mice with DCM on a C57BL/6J background. DCM mice express a cardiomyocyte-specific dominant-negative CREB transcription factor, and reproducibly progress through Stages A-D of human HF, although kidney function remains within a normal range. Mice were randomly assigned to an ad-libitum maintenance of identical diets except for sodium content: a common laboratory diet for mice of 0.3% sodium (NSD, Envigo Teklad 7912; Madison, Wis., USA) or low 0.05% sodium diet (LSD, custom ordered based on Envigo Teklad 7034; Madison, Wis., USA). Diets were otherwise similar in other components, including potassium (7912—0.8% and 7043—0.9%). Diets were initiated from 28 days of age corresponding to stage A HF and maintained until experimental end-point or natural death. A specific number of mice in each subgroup were randomly designated for survival studies or terminal end-point at 13 (stage C HF), 17 and 20 (stage D HF) weeks of age for tissue and blood collection (via cardiocentesis in prepared EDTA-aprotinin syringes to block coagulation and proteolysis) as previously reported. Mice were euthanized with an overdose of inhaled 5% isoflurane (IsoFlo, Zoetis Inc., United Kingdom) and death was confirmed by the absence of respiration and heartbeat.

DCM male littermate mice were randomly assigned to a low sodium diet (LSD) (treatment) or normal sodium diet (NSD) (control) initiated from 28 days of age corresponding to stage A HF and maintained long-term as mice progressed through A to D HF stages for ≥13 weeks as mentioned above. Littermate mice without DCM (wild type, WT) were used as a control for DCM-HF progression.

Echocardiographic examination of sub-groups of DCM mice at 140 days of age (Stage D HF) detected fluid motion in the chest of the pleural effusion of DCM mice on a NSD but not on a LSD. The necropsy assessment confirmed echocardiographic observations and revealed that ˜60% of mice on a NSD developed pleural effusions (edema fluid accumulation in the thoracic cavity outside the lung caused by fluid movement from pulmonary edema across the visceral pleura), while no mice on a LSD had pleural effusions (p<0.01; FIG. 7A). Similarly, lung weights were significantly increased in mice on a NSD (p<0.001) when compared with WT controls (FIG. 7B) on a NSD, consistent with increased pulmonary fluid retention (pulmonary congestion, alveolar and intra-alveolar edema identified by lung histology, chest radiography and cardiac magnetic resonance imaging/MRI). Lung weights were significantly reduced in mice on a LSD vs. NSD (p<0.05; FIG. 2B).

The effect of a LSD on systemic water retention was assessed by measuring total body extracellular water (ECW) or free water, using noninvasive quantitative magnetic resonance (QMR) for body composition monitoring. DCM mice on a NSD had significantly increased ECW when compared to WT littermate controls at 13 (Stage C HF, p<0.05) and 17 (Stage D HF, p<0.01) weeks of age (FIG. 7C). DCM mice on a LSD roughly a 5.5-fold decrease vs. DCM group on NSD (p<0.001; FIG. 7C). There was no difference in ECW values up to 17 weeks of age in DCM mice on a LSD vs. WT littermate controls. Body weights (FIG. 7D) and total water measurements (FIG. 7E) were relatively comparable among all groups.

Consistent with its effect on decreasing the development of edema and effusions, a LSD significantly increased median survival in DCM mice by comparison to a NSD (23 vs. 20 weeks, P<0.001; FIG. 7F).

### Example 7

### Longitudinal Measurements of ECW and Treatment Strategy for Changes in ECW

A 49-year-old male patient has his annual physical from his primary care physician. In addition to the standard test and physical exam, his physician recommends that the man also gets his extracellular water (ECW), muscle/lean mass, and fat measured with a quantitative magnetic resonance (QMR) machine. This establishes baseline levels of the patient.

One year later, the patient comes to get his yearly physical. During the exam, the physician notices that the patient's ECW level has increased by 15% compared to the ECW level measurement of his baseline, which concerns the physician. The physician orders more testing and the results show that the patient has a heart condition. The physician prescribes a drug treatment to the patient and asks that he return in 6 months for a follow-up appointment. After six months, the patient's ECW levels are measured again, unfortunately, no progress has been made in lowering his ECVV levels. The physician decides to change the patient's treatment by increasing the drug dosage and again asks that he returns in 6 months for a follow-up appointment. Again, the patient returns after 6 months to have his ECW levels measured. The physician determines that with this treatment the ECW levels have decreased by 5%. Pleased with this response, the physician recommends that the patient remains on this treatment, with follow-up appointments every 6 months. After 1.5 years of treatment, the man's ECW levels have normalized back to his baseline.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed is:

1. A method for treating a patient who is suffering from a heart condition, the method comprising the steps of:
a. determining whether the patient has extracellular water (ECW) retention by:
i. performing quantitative magnetic resonance (QMR) to measure extracellular water content of said patient; and
ii. determining a status of the heart condition in said patient or risk of death of said patient based on measured ECW content; and
b. if the patient has an increase of ECW retention by more than 5% compared to a baseline level, administering a treatment to the patient.

2. The method of claim 1, wherein the heart condition comprises heart failure (HF), HF-associated and non-associated -sarcopenia/cachexia, -necrosis, -liver disease, and/or -kidney disease, HF-reduced ejection fraction (HFrEF), HF-preserved ejection fraction (HFpEF), or heart dysfunction.

3. The method of claim 1, wherein a baseline level comprises ECW levels from a healthy subject, wherein a healthy subject comprises a subject without said heart condition.

4. The method of claim 1, wherein the baseline level comprises ECW levels previously measured from the patient at a time of initial presentation or diagnosis of said heart condition.

5. The method of claim 1, wherein said treatment comprises therapeutically effective drugs or intervention that modulate extracellular water content

6. The method of claim 5, wherein said therapeutically effective drugs comprise direct and indirect renin inhibitors and inhibitors to the renin pathway, which result in decrease production of active renin, diuretics, dobutamine, epinephrine, norepinephrine, and therapeutics against angiotensin, aldosterone, angiotensin converting enzyme or neprilysin, beta blockers, angiotensin receptor blockers, antiarrhythmic agents, anticoagulants, cholesterol-lowering drugs, statins, and/or digoxin.

7. The method of claim 5, wherein said intervention comprises the use of direct renin inhibitors in combination with diuretics, dobutamine, and therapeutics against angiotensin, aldosterone or neprilysin, beta blockers, antiarrhythmic agents, anticoagulants, cholesterol-lowering drugs, and/or digoxin, additional and/or more frequent monitoring, physically draining a cavity, exercising or nutritional alterations.

8. The method of claim 1, wherein said treatment further comprises devices that improve or stabilize cardiac function comprising pacemakers, defibrillators, circulatory assistance, artificial hearts, and/or transplantation.

9. A method of monitoring effectiveness of a treatment in a patient with a heart condition, wherein the treatment is currently being administered to said patient, comprising the steps of:
a. performing quantitative magnetic resonance (QMR) to measure extracellular water content of said patient;
b. determining a status of the heart condition in said patient or risk of death of said patient based on measured extracellular water content; and
c. determining changes in ECW retention compared to a baseline level based on measured extracellular water content,
wherein if the patient has an increase of extracellular water retention by more than 5% compared to the baseline level, a different treatment is administered to the patient;
wherein if the patient has a decrease of extracellular water retention by more than 5% compared to a baseline level, the patient maintains the current treatment.

10. The method of claim 9, wherein the heart condition comprises heart failure (HF), HF-associated and non-associated -sarcopenia/cachexia, -necrosis, -liver disease, and/or -kidney disease, HF-reduced ejection fraction (HFrEF), HF-preserved ejection fraction (HFpEF), or heart dysfunction.

11. The method of claim 9, wherein the baseline level comprises ECW levels from a healthy subject, wherein a healthy subject comprises a subject without said heart condition.

12. The method of claim 9, wherein the baseline level comprises ECW levels previously measured from the patient at a time of initial presentation or diagnosis of said heart condition.

13. The method of claim 9, wherein said treatment comprises therapeutically effective drugs or intervention that modulate extracellular water content.

14. The method of claim 13, wherein said therapeutically effective drugs comprise direct and indirect renin inhibitors and inhibitors to the renin pathway, which result in decrease production of active renin, diuretics, dobutamine, epinephrine, norepinephrine, and therapeutics against angiotensin, aldosterone, angiotensin converting enzyme or neprilysin, beta blockers, angiotensin receptor blockers, antiarrhythmic agents, anticoagulants, cholesterol-lowering drugs, statins, and/or digoxin.

15. The method of claim 13, wherein said intervention comprises the use of direct renin inhibitors in combination with diuretics, dobutamine, and therapeutics against angiotensin, aldosterone or neprilysin, beta blockers, antiarrhythmic agents, anticoagulants, cholesterol-lowering drugs (e.g., statins), and/or digoxin, additional and/or more frequent monitoring, physically draining a cavity, exercising or nutritional alterations.

16. The method of claim 9, wherein said treatment further comprises devices that improve or stabilize cardiac function comprising pacemakers, defibrillators, circulatory assistance, artificial hearts, and/or transplantation.
